(19) United States
(12) Patent Application Publication
(10) Pub. No.: US 2025/0001026 A1
(43) Pub. Date: Jan. 2, 2025

(54) Decontamination Apparatus

(71) Applicant: LiViliti Health Products, Lake City, FL (US)

(72) Inventors: Brian Sharpe, Irmo, SC (US); James Gilkison, Bradenton, FL (US); Caleb Umstead, Lake City, FL (US)

(73) Assignee: LiViliti Health Products, Lake City, FL (US)

(21) Appl. No.: 18/756,935

(22) Filed: Jun. 27, 2024

Related U.S. Application Data
(60) Provisional application No. 63/511,278, filed on Jun. 30, 2023.

(51) Int. Cl. A61L 2/10; A61L 2/26

(52) U.S. Cl. CPC A61L 2/10; A61L 2/26; A61L 2202/11; A61L 2202/122; A61L 2202/24

(57) ABSTRACT

An apparatus for decontaminating a target object includes a body defining a decontamination chamber, a plurality of decontaminating light elements to emit decontaminating radiation in the decontamination chamber, and a transmissive mounting structure for supporting the target object in the decontamination chamber, wherein the transmissive mounting structure comprises quartz glass.

### RELATED APPLICATION

This application claims priority to commonly owned U.S. Provisional Patent Application No. 63/511,278 filed Jun. 30, 2023, the entire contents of which are hereby incorporated by reference for all purposes.

### TECHNICAL FIELD

The present invention relates to a decontamination apparatus, for example for treating breathing masks (e.g., CPAP masks) or other medical devices.

### BACKGROUND

Medical devices, for example CPAP masks and other breathing masks, are periodically decontaminated for repeated use. Conventional systems for decontaminating medical devices often include a container in which one or more medical devices may be arranged and treated. However, because medical devices vary in size and shape, conventional decontamination systems often fail to effectively treat all areas (e.g., all surface areas) of at least some devices in the container, especially medical devices with irregular or unique shapes, such as a typical breathing mask (e.g., CPAP mask).

Incomplete decontamination of a contaminated medical device may cause various problems, for example infection or other illness of the wearer. Accordingly, there is a need for a decontamination apparatus (for medical devices and/or other devices) that effectively decontaminates the relevant areas (e.g., surface areas) of respective devices.

### SUMMARY

The present disclosure provides a decontamination apparatus for decontaminating target objects, wherein target objects may include breathing masks (e.g., CPAP masks, BiPAP masks, oxygen masks, hospital respiratory masks, nebulizer masks, non-invasive ventilation masks, laryngeal mask, etc.) nasal pillows (e.g., CPAP or BiPAP nasal pillows), other medical devices and/or other non-medical devices to be decontaminated.

One aspect provides an apparatus for decontaminating a target object. The apparatus includes a body defining a decontamination chamber, a plurality of decontaminating light elements to emit decontaminating radiation in the decontamination chamber, and a transmissive mounting structure for supporting the target object in the decontamination chamber, wherein the transmissive mounting structure comprises quartz glass.

In some embodiments, the plurality of decontaminating light elements comprise UVC (ultraviolet-C) light elements.

In some embodiments, the plurality of decontaminating light elements comprise UV bulbs or lamps comprising mercury.

In some embodiments, the plurality of decontaminating light elements includes at least one decontaminating light element integrated in or affixed to the transmissive mounting structure.

In some embodiments, the transmissive mounting structure comprises a platform to support the target object above a base of the body, and the plurality of decontaminating light elements includes at least one decontaminating light element located below the transmissive mounting structure, wherein the transmissive mounting structure allows transmission of decontaminating radiation from the at least one decontaminating light element to the target object.

In some embodiments, the transmissive mounting structure comprises a transmissive post extending from a base, a sidewall, or a lid of the body.

In some embodiments, the plurality of decontaminating light elements includes at least one decontaminating light element integrated in or affixed to the transmissive post.

In some embodiments, the transmissive post has an elongated shape extending along a first direction from with a base end to a distal end, the distal end configured to support the target object, and the plurality of decontaminating light elements includes at least one decontaminating light element arranged to emit decontaminating radiation within the transmissive post along the first direction such that the decontaminating radiation is internally reflected in the transmissive post along the first direction and radiates from the distal end of the transmissive post.

In some embodiments, the apparatus is configured to achieve log 2 decontamination of at least one contaminant organism.

In some embodiments, the apparatus is configured to achieve log 3 decontamination of at least one contaminant organism.

In some embodiments, the apparatus is configured to achieve log 4 decontamination of at least one contaminant organism.

In some embodiments, the apparatus is configured to achieve log 5 decontamination of at least one contaminant organism.

Another aspect provides an apparatus for decontaminating a target object. The apparatus includes a body including a base, a sidewall, and a lid movable between an open position and a closed position, wherein the body defines a decontamination chamber for receiving a target object, a mounting structure extending from the base for supporting the target object spaced apart from the body, and a plurality of decontaminating light elements to decontaminate the target object supported by the mounting structure.

In some embodiments, the mounting structure comprises a cone-shaped structure.

In some embodiments, the plurality of decontaminating light elements includes at least one decontaminating light element provided on the mounting structure.

In some embodiments, the plurality of decontaminating light elements includes a plurality of decontaminating light elements provided on the body.

In some embodiments, the plurality of decontaminating light elements comprise UVC (ultraviolet-C) light elements.

In some embodiments, the plurality of decontaminating light elements comprise UV bulbs or lamps comprising mercury.

### BRIEF DESCRIPTION OF THE DRAWINGS

Example aspects of the present disclosure are described below in conjunction with the figures, in which:

FIGS. 1A and 1B show a three-dimensional view from above (FIG. 1A) and a cross-sectional side view (FIG. 1B) of an example decontamination apparatus including a transmissive platform for supporting a target object, according to one embodiment;

FIGS. 2A and 2B show a three-dimensional view from above (FIG. 2A) and a cross-sectional side view (FIG. 2B) of an example decontamination apparatus including a transmissive post for supporting a target object, according to one embodiment;

FIGS. 3A and 3B show three-dimensional views from above of an example decontamination apparatus including a support post without a target object present (FIG. 3A) and with a target object arranged on the support post (FIG. 3B), according to one embodiment; and

FIGS. 4A-4C show three example mounting structures that may be used to support a target object in a decontamination apparatus.

It should be understood that the reference number for any illustrated element that appears in multiple different figures has the same meaning across the multiple figures, and the mention or discussion herein of any illustrated element in the context of any particular figure also applies to each other figure, if any, in which that same illustrated element is shown.

### DETAILED DESCRIPTION

Embodiments of the present invention provide a decontamination apparatus for decontaminating target objects, wherein target objects may include breathing masks (e.g., CPAP masks, BiPAP masks, oxygen masks, hospital respiratory masks, nebulizer masks, non-invasive ventilation masks, laryngeal mask, etc.) nasal pillows (e.g., CPAP or BiPAP nasal pillows), other medical devices and/or other non-medical devices to be decontaminated.

As used herein, “decontaminating” may include cleaning, sanitizing, disinfecting, sterilizing, and/or dehumidifying, which may include killing, damaging, or effectively inactivating bacteria, viruses, mold, and/or other microorganisms. In some examples, decontamination may include killing, damaging, or effectively inactivating one or more of the following types of organisms, collectively referred to herein as the “Example Contaminant Organisms”: (a) Bacillus species (e.g., Bacillus atrophaeus), (2) Coagulase-negative staphylococcus (e.g., Staphylococcus epidermidis), (3) Methicillin-resistant Staphylococcus aureus and Staphylococcus aureus (e.g., Staphylococcus aureus), (4) Micrococcus species (e.g., Micrococcus luteus), and/or (5) Diphtheroid representative (e.g., Corynebacterium xerosis).

For example, in some embodiments, the decontamination apparatus may be configured to achieve “log 2 decontamination” of at least 1, at least 2, at least 3, at least 4, or all 5 (depending on the embodiment) of the Example Contaminant Organisms, wherein log 2 decontamination of each respective Example Contaminant Organism means killing, damaging, or effectively inactivating at least 99% (log 2) of the respective Example Contaminant Organism on the target object.

For example, in some embodiments, the decontamination apparatus may be configured to achieve “log 3 decontamination” of at least 1, at least 2, at least 3, at least 4, or all 5 (depending on the embodiment) of the Example Contaminant Organisms, wherein log 3 decontamination of each respective Example Contaminant Organism means killing, damaging, or effectively inactivating at least 99.9% (log 3) of the respective Example Contaminant Organism on the target object.

For example, in some embodiments, the decontamination apparatus may be configured to achieve “log 4 decontamination” of at least 1, at least 2, at least 3, at least 4, or all 5 (depending on the embodiment) of the Example Contaminant Organisms, wherein log 4 decontamination of each respective Example Contaminant Organism means killing, damaging, or effectively inactivating at least 99.99% (log 4) of the respective Example Contaminant Organism on the target object.

For example, in some embodiments, the decontamination apparatus may be configured to achieve “log 5 decontamination” of at least 1, at least 2, at least 3, at least 4, or all 5 (depending on the embodiment) of the Example Contaminant Organisms, wherein log 5 decontamination of each respective Example Contaminant Organism means killing, damaging, or effectively inactivating at least 99.999% (log 5) of the respective Example Contaminant Organism on the target object.

For example, in some embodiments, the decontamination apparatus may be configured to achieve “log 6 decontamination” of at least 1, at least 2, at least 3, at least 4, or all 5 (depending on the embodiment) of the Example Contaminant Organisms, wherein log 6 decontamination of each respective Example Contaminant Organism means killing, damaging, or effectively inactivating at least 99.9999% (log 6) of the respective Example Contaminant Organism on the target object.

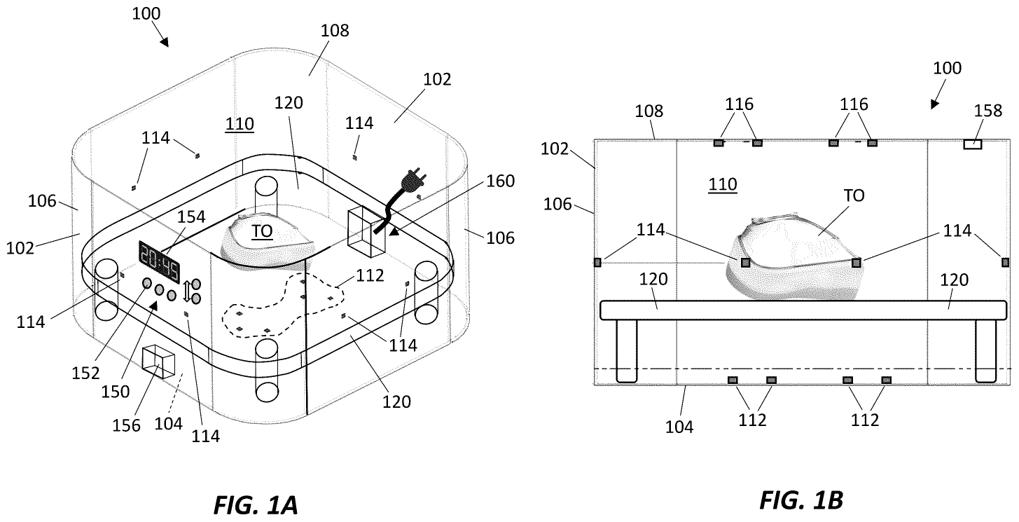

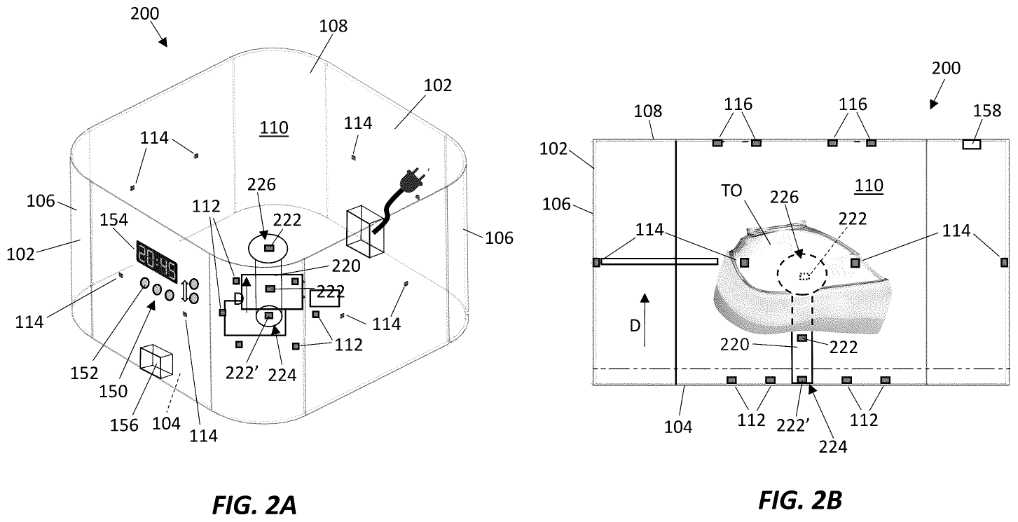

In some examples, e.g., as shown in FIGS. 1A-1B and 2A-2B discussed below, the decontamination apparatus may include a body defining a decontamination chamber, a plurality of decontaminating light elements to emit decontaminating radiation in the decontamination chamber, and a transmissive mounting structure for supporting at least one target object in the decontamination chamber. For convenience, the following discussion refers to (and the drawings show) a single target object arranged in the decontamination chamber; however, it should be understood that multiple target objects may be arranged in the decontamination chamber for simultaneous decontamination.

The transmissive mounting structure may comprise quartz glass, which is transparent to certain radiation, including UVC light. In some embodiments, quartz glass may comprise fused silica or fused quartz, i.e., a glass including nearly pure silica in amorphous form. In some embodiments, the transmissive mounting structure comprises a comprise a raised platform on which a target object may be supported (e.g., a platform raised above a base (or floor) of the body to allow UVC irradiation of the target object from below), or an elongated structure (e.g., a cone, post, or pillar on which a target objects may be supported (e.g., to allow UVC irradiation of interior surfaces of the target object).

The decontaminating light elements in any embodiment discussed herein may include UVC LEDs. UVC light (radiation in the wavelength range of 200-280 nm) is a known disinfectant for air, water, and non-porous surfaces, for example in the 250-270 nm range. For example, UVC exposure may induce damage to the genetic material of microorganisms, including the Example Contaminant Organisms, other microorganisms, and/or viruses, to render them inactive. Accordingly, the decontamination apparatus may include UVC LEDs to emit radiation in the 200-280 nm range, for example in the 250-280 nm range, and in some embodiments in the in the 260-265 nm range. Alternatively, the decontaminating light elements in any embodiment discussed herein may comprise UV bulbs or lamps comprising mercury (“mercury UV bulbs”). Accordingly, any embodiment discussed herein including UVC LEDs may alternatively use mercury UV bulbs, or a combination of UVC LEDs and mercury UV bulbs.

FIGS. 1A and 1B show a three-dimensional view from above (FIG. 1A) and a cross-sectional side view (FIG. 1B) of an example decontamination apparatus 100 for decontaminating a target object TO according to one embodiment of the present invention. In this example the target object TO may comprise a breathing mask. For illustrative purposes only, the target object TO is shown in FIG. 1B but not in FIG. 1A.

As shown, the example decontamination apparatus 100 includes a body 102 including a base 104, sidewalls 106, and a lid 108 defining a decontamination chamber 110. One or more arrays of UVC LEDs may be located in or on the body 102, for example in or on the base 104, sidewall(s) 106, and/or lid 108. In the example shown in FIGS. 1A-1B, the decontamination apparatus 100 includes a first UVC LED array 112 arranged on the base 104, a second UVC LED array 114 arranged on the sidewalls 106 around the lateral perimeter of the body 102, and a third UVC LED array 116 arranged on the lid 108.

A transmissive platform 120 (or a rack or shelf) for supporting the target object TO may be fixedly or removably arranged in the decontamination chamber 110. The transmissive platform 120 may be formed from quartz glass, which is transparent to UVC radiation emitted by LED arrays 112, 114, and 116. In some embodiments, the transmissive platform 120 may comprise a quartz glass plate having a vertical thickness in the range of 0.1 to 5.0 cm thickness, and in particular embodiments, a vertical thickness in the range of 0.2 to 0.9 cm, which has indicated effective performance in certain prototype testing. In some embodiments (e.g., as shown in FIGS. 1A-1B), the transmissive platform 120 may be raised above the base 104 by feet or other structures. Alternatively, the transmissive platform 120 may extend from a sidewall 106 of the decontamination apparatus 100, e.g., with a shelf type arrangement.

In some embodiments, the transmissive platform 120 may support, or “suspend,” a target object TO (or multiple target objects TO) in a manner that substantially or fully eliminates “shadows,” i.e., non-illuminated areas of the target object TO, thereby allowing irradiation of all surfaces of the target object TO, including at areas of contact between the target object TO and transmissive platform 120.

As shown in FIG. 1A, the decontamination apparatus 100 may include a user interface 150, control circuitry 156, and a power supply 160 to provide power to the user interface 150, control circuitry 156, and LEDs 112, 114, and 116. The power supply 160 may include circuitry to interface with a power grid, or alternatively may include a battery and associated circuitry. The user interface 150 may include user input devices 152 and a display 154. The user input devices 152 may include buttons, sliders, switches, and/or other devices allowing a user to input and/or adjust various operational settings of the decontamination apparatus 100, for example (a) an input device to power the apparatus 100 on/off, (b) input device(s) to select a decontamination cycle time, and/or (c) input device(s) to select an operating intensity, e.g., by controlling the power supplied to respective LEDs 112, 114, and 116.

The display 154 may display various information regarding the status or operation of the decontamination apparatus 100, for example (a) a user selected decontamination cycle time and/or operating intensity, (b) an indication that the decontamination apparatus 100 is operating (e.g., LEDs 112, 114, and 116 are powered up), (c) a remaining time of an ongoing decontamination cycle, (c) a cumulative usage time (e.g., total hours of use), (d) a notification that a decontamination cycle has completed, (e) a position of the lid 108 (e.g., based on signals from a lid position switch 158 as discussed below), and/or (f) an error message. In some embodiments, decontamination apparatus 100 may include a speaker to output audible notifications, e.g., to indicate any of the types of information listed above.

The control circuitry 156 may include any circuitry to control the operation of the decontamination apparatus 100, e.g., based on input received via user input devices 152. The control circuitry 156 may include a processor, memory device(s), and logic instructions (e.g., embodied in software and/or firmware) stored in the memory device(s) and executable by the processor to control the operation the decontamination apparatus 100, for example to enable/disable respective LEDs 112, 114, and 116, control the intensity emitted by respective LEDs 112, 114, and 116, control the information displayed via display 154, and/or identify error conditions and generate alert notifications.

In some embodiments, decontamination apparatus 100 may include a lid position switch 158 (see FIG. 1B) connected to control circuitry 156, to detect whether the lid 108 is open or closed. In some embodiments, the control circuitry 156 enables operation of the decontamination apparatus 100 when the lid position switch 158 indicates the lid 108 is closed, and disables operation of the decontamination apparatus 100 (e.g., by interrupting power delivery to LEDs 112, 114, and 116) when the lid position switch 158 indicates the lid 108 is open, e.g., to protect a user from radiation.

In some embodiments, UVC LEDs 112, 114, and/or 116, powered by power supply 160 and controlled by control circuitry 156, are configured to collectively deliver to respective surfaces of the target object TO a UV intensity in the range of 10-5000 μW/cm² for a cycle time duration in the range of 10 seconds to 30 minutes. For example, in some embodiments UVC LEDs 112, 114, and/or 116 are configured to collectively deliver to respective surfaces of the target object TO a UV intensity in the range of 10-500 μW/cm² for a cycle time duration in the range of 1 minute to 30 minutes. As another example, in some embodiments UVC LEDs 112, 114, and/or 116 are configured to collectively deliver to respective surfaces of the target object TO a UV intensity in the range of 500-5000 μW/cm² for a cycle time duration in the range of 10 seconds to 5 minutes.

In some embodiments, the example decontamination apparatus 100 may achieve log 2 decontamination, log 3 decontamination, log 4 decontamination, log 5 decontamination, or log 6 decontamination, for example depending on the number, intensity and arrangement of UVC LEDS 112, 114, and/or 116.

FIGS. 2A and 2B show a three-dimensional view from above (FIG. 2A) and a cross-sectional side view (FIG. 2B) of another example decontamination apparatus 200 for decontaminating a breathing mask according to another embodiment. The decontamination apparatus 200 is similar to example decontamination apparatus 100 shown in FIGS. 1A-1B, but includes a transmissive post 220 for supporting a target object TO in the decontamination chamber 110 rather than the transmissive platform 120 discussed above. The transmissive post 220 may have an elongated shape extending along a first direction D from with a base end 224 to a distal end 226, wherein the distal end 226 is configured to support the target object TO. In other embodiments, the transmissive post 420 (on which a target object TO may be mounted) may extend in another direction from a sidewall 106 or the lid 108 of the body 102, rather than the base 104. At least a portion of the transmissive post 220 may be formed from quartz glass, which is transparent to UVC radiation, e.g., UVC radiation emitted by LED arrays 112, 114, and 116. As used herein, a “post” may comprise any elongated structure, e.g., having a shape of an elongated cone, pillar, pedestal, arm, etc.

In some embodiments, one or more UVC LED 222 may (optionally) be mounted on, integrated with or arranged adjacent or proximate the transmissive post 220, e.g., to effectively apply UVC radiation to interior surfaces of the target object TO. In some embodiments, one or more UVC LED 222, e.g., indicated in FIG. 2B as UVC LED 222′, may be arranged to propagate UVC radiation along the elongated length of the post 220 in the first direction D, wherein the UVC radiation (or at least a portion thereof) is internally reflected in the transmissive post 220 along the first direction D and radiates from the distal end 226 of the transmissive post 220 on which the target object TO is supported, e.g., to irradiate interior surfaces of the target object TO. For example, as shown in FIGS. 2A-2B, a UVC LED 222′ may be located or near at the base end 224 of the transmissive post 220 to emit radiation propagating along (and internally reflected within) the transmissive post 220 in the first direction D and radiating out from the transmissive post 220 at the distal end 226.

Like the decontamination apparatus 100, the example decontamination apparatus 200 may include user interface 150, control circuitry 156, power supply 160, and lid position switch 158 as discussed above. In some embodiments, UVC LEDs 112, 114, and/or 116 of decontamination apparatus 200, powered by power supply 160 and controlled by control circuitry 156, are configured to collectively deliver to respective surfaces of the target object TO a UV intensity in the range of 10-5000 μW/cm² for a cycle time duration in the range of 10 seconds to 30 minutes. For example, in some embodiments UVC LEDs 112, 114, and/or 116 are configured to collectively deliver to respective surfaces of the target object TO a UV intensity in the range of 10-500 μW/cm² for a cycle time duration in the range of 1 minute to 30 minutes. As another example, in some embodiments UVC LEDs 112, 114, and/or 116 are configured to collectively deliver to respective surfaces of the target object TO a UV intensity in the range of 500-5000 μW/cm² for a cycle time duration in the range of 10 seconds to 5 minutes.

In some embodiments, the example decontamination apparatus 200 may achieve log 2 decontamination, log 3 decontamination, log 4 decontamination, log 5 decontamination, or log 6 decontamination, for example depending on the number, intensity and arrangement of UVC LEDs 112, 114, 116, 222, and/or 222′.

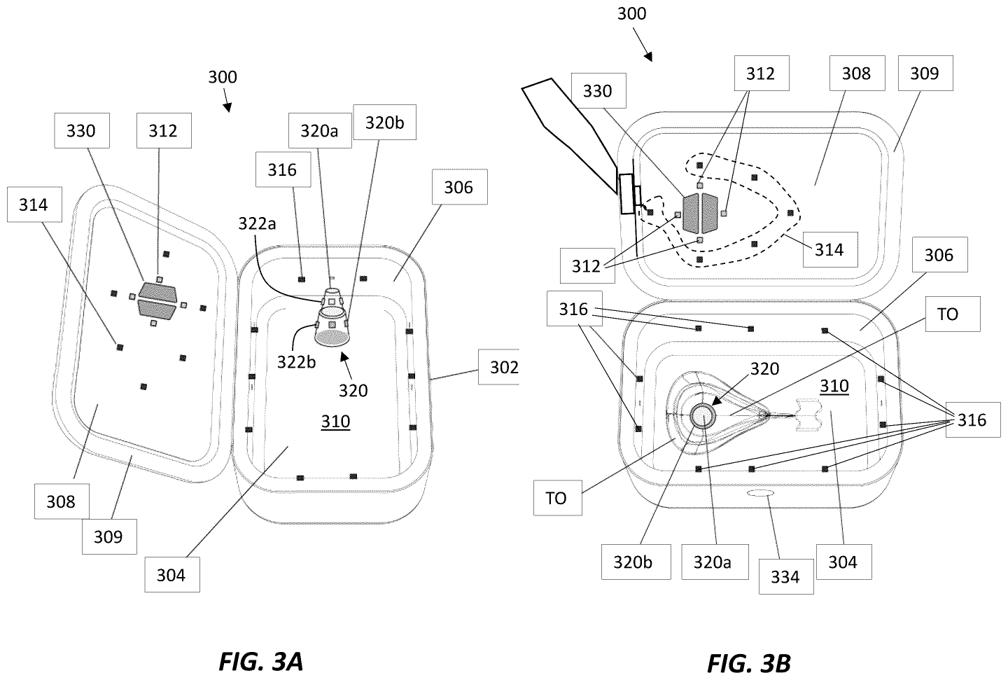

FIGS. 3A and 3B show three-dimensional views from above of an example decontamination apparatus 300 including a support post 320 (e.g., a support cone) without a target object TO arranged thereon (FIG. 3A) and with a target object TO arranged thereon (FIG. 3B), according to one embodiment. As shown, the example decontamination apparatus 300 includes a body 302 including a base 304, sidewalls 306, and a lid 308 defining a decontamination chamber 310.

One or more arrays of UVC LEDs may be located in or on the body 302, for example in or on the base 304, sidewall(s) 306, and/or lid 308. In the example shown in FIGS. 3A-3B, the decontamination apparatus 300 includes a first UVC LED array 312 arranged on the sidewalls 306, a second UVC LED array 314 arranged on the lid 308 at a location above the support post 320, and a third UVC LED array 316 arranged on the lid 108 in a pattern extending around the second UVC LED array 314.

The decontamination apparatus 300 may also include a fan 330 located on the lid 308 to fan creates air flow around and/or through the target object TO, e.g., for dehumidifying the target object TO and increasing decontamination effectiveness, for example by blowing bacteria, mold, and other microorganisms off the target object TO to be killed or inactivated by the UVC radiation.

The first UVC LED array 312 (e.g., including 4 UVC LEDs) may be configured to focus on an interior of the target object TO, e.g., an interior of a breathing mask. LED array 102 may have a different focal point or angle than the second UVC LED array 314 and third UVC LED array 316, e.g., to have more focused penetration in the interior of the target object TO.

The second UVC LED array 314 (e.g., including 6 UVC LEDs) may be configured to focus on a perimeter of the target object TO, e.g., a perimeter of a face cushion 112.

The third UVC LED array 316 (e.g., including 10 UVC LEDs) may be configured to focus on a perimeter of the target object TO, as well as any microbial particles that may be blown around in the decontamination chamber 310 by the fan 106.

The support post 320 (e.g., support cone) may include an upper post portion (e.g., upper cone) 320a and a lower post portion (e.g., lower cone) 320b, wherein one or more UVC LEDs may be located on the upper post portion 320a and/or lower post portion 320b as discussed below. The upper post portion 320a may be shaped and sized to support certain types of target objects TO, for example, nasal pillows or nasal masks, and may include a UVC LED array 322a, for example including three 120 degree LEDS targeting 360 degrees of the interior of the target object TO (e.g., nasal pillows). The lower post portion 320b may be shaped and sized to support other types of target objects TO, for example, full face masks (e.g., CPAP full face masks), and may include a UVC LED array 322b, for example including three 120 degree LEDS targeting 360 degrees of the interior of the target object TO (e.g., full face mask).

In some examples, reflectors may be affixed or otherwise provide on respective interior surfaces base 304, sidewalls 306, and/or lid 308 facing the decontamination chamber 310, for reflecting LED radiation, e.g., emitted by any of UVC LED array 312, 314, 316, 322a, and/or 322b.

In some examples, the lid 308 may include a transparent perimeter rim 309 allowing a user to visually determine if the decontamination apparatus 300 is in operation without risk of UVC light exposure to the user.

Like the decontamination apparatus 100 and 200, the example decontamination apparatus 300 may include a user interface, control circuitry, power supply, and lid position switch corresponding with the user interface 150, control circuitry 156, power supply 160, and lid position switch 158 discussed above. In some embodiments, UVC LEDs 312, 314, 316, 322a, and/or 322b′, powered by the power supply and controlled by respective control circuitry, are configured to collectively deliver to respective surfaces of the target object TO a UV intensity in the range of 10-5000 μW/cm² for a cycle time duration in the range of 10 seconds to 30 minutes. For example, in some embodiments UVC LEDs 312, 314, 316, 322a, and/or 322b′ are configured to collectively deliver to respective surfaces of the target object TO a UV intensity in the range of 10-500 μW/cm² for a cycle time duration in the range of 1 minute to 30 minutes. As another example, in some embodiments UVC LEDs 312, 314, 316, 322a, and/or 322b′ are configured to collectively deliver to respective surfaces of the target object TO a UV intensity in the range of 500-5000 μW/cm² for a cycle time duration in the range of 10 seconds to 5 minutes.

In some embodiments, the example decontamination apparatus 300 may achieve log 2 decontamination, log 3 decontamination, log 4 decontamination, log 5 decontamination, or log 6 decontamination, for example depending on the number, intensity and arrangement of UVC LEDS 312, 314, 316, 322a, and/or 322b′.

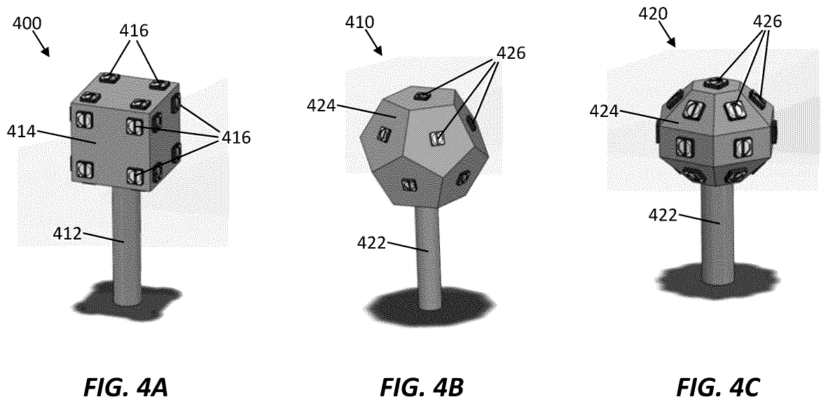

FIGS. 4A-4C show three example mounting structures that may be used in any of the example decontamination apparatuses discussed above, for supporting a target object, e.g., a breathing mask. FIG. 4A shows a first example mounting structure 400 comprising a post 402 with a cube shaped head 404 having an array of UVC LEDs 406 mounted thereon. FIG. 4B shows a second example mounting structure 410 comprising a post 412 with a dodecahedron shaped head 414 having an array of UVC LEDs 416 mounted thereon. FIG. 4C shows a third example mounting structure 420 comprising a post 422 with a ball shaped head 424 having an array of UVC LEDs 426 mounted thereon.

Although example embodiments have been described above, other variations and embodiments may be made from this disclosure without departing from the spirit and scope of these embodiments.

1. An apparatus for decontaminating a target object, the apparatus comprising:
a body defining a decontamination chamber;
a plurality of decontaminating light elements to emit decontaminating radiation in the decontamination chamber; and
a transmissive mounting structure for supporting the target object in the decontamination chamber, the transmissive mounting structure comprising quartz glass.

2. The apparatus of claim 1, wherein the plurality of decontaminating light elements comprise UVC (ultraviolet-C) light elements.

3. The apparatus of claim 1, wherein the plurality of decontaminating light elements comprise UV bulbs or lamps comprising mercury.

4. The apparatus of claim 1, wherein the plurality of decontaminating light elements includes at least one decontaminating light element integrated in or affixed to the transmissive mounting structure.

5. The apparatus of claim 1, wherein:
the transmissive mounting structure comprises a platform to support the target object above a base of the body; and
the plurality of decontaminating light elements includes at least one decontaminating light element located below the transmissive mounting structure, wherein the transmissive mounting structure allows transmission of decontaminating radiation from the at least one decontaminating light element to the target object.

6. The apparatus of claim 1, wherein the transmissive mounting structure comprises a transmissive post extending from a base, a sidewall, or a lid of the body.

7. The apparatus of claim 6, wherein the plurality of decontaminating light elements includes at least one decontaminating light element integrated in or affixed to the transmissive post.

8. The apparatus of claim 6, wherein:
the transmissive post has an elongated shape extending along a first direction from with a base end to a distal end, the distal end configured to support the target object; and
the plurality of decontaminating light elements includes at least one decontaminating light element arranged to emit decontaminating radiation within the transmissive post along the first direction such that the decontaminating radiation is internally reflected in the transmissive post along the first direction and radiates from the distal end of the transmissive post.

9. The apparatus of claim 1, wherein the apparatus is configured to achieve log 2 decontamination of at least one contaminant organism.

10. The apparatus of claim 1, wherein the apparatus is configured to achieve log 3 decontamination of at least one contaminant organism.

11. The apparatus of claim 1, wherein the apparatus is configured to achieve log 4 decontamination of at least one contaminant organism.

12. The apparatus of claim 1, wherein the apparatus is configured to achieve log 5 decontamination of at least one contaminant organism.

13. An apparatus for decontaminating a target object, the apparatus comprising:
a body including a base, a sidewall, and a lid movable between an open position and a closed position;
wherein the body defines a decontamination chamber for receiving a target object;
a mounting structure extending from the base for supporting the target object spaced apart from the body; and
a plurality of decontaminating light elements to decontaminate the target object supported by the mounting structure.

14. The apparatus of claim 13, wherein the mounting structure comprises a cone-shaped structure.

15. The apparatus of claim 13, wherein the plurality of decontaminating light elements includes at least one decontaminating light element provided on the mounting structure.

16. The apparatus of claim 13, wherein the plurality of decontaminating light elements includes a plurality of decontaminating light elements provided on the body.

17. The apparatus of claim 13, wherein the plurality of decontaminating light elements comprise UVC (ultraviolet-C) light elements.

18. The apparatus of claim 13, wherein the plurality of decontaminating light elements comprise UV bulbs or lamps comprising mercury.
